(19) United States
(12) Patent Application Publication
(10) Pub. No.: US 2025/0034208 A1
(43) Pub. Date: Jan. 30, 2025

(54) PEPTIDES AND PHARMACEUTICAL AND COSMETIC COMPOSITIONS CONTAINING THEM

(71) Applicants: LABORATOIRES D'ANJOU, Paris (FR); CENTRE NATIONAL DE LA RECHERCHE SCIENTIFIQUE- CNRS, Paris (FR); UNIVERSITÉ CLAUDE BERNARD LYON 1, Villeurbanne (FR)

(72) Inventor: Patricia ROUSSELLE, Lyon (FR)

(73) Assignees: LABORATOIRES D'ANJOU, Paris (FR); CENTRE NATIONAL DE LA RECHERCHE SCIENTIFIQUE-CNRS, Paris (FR); UNIVERSITÉ CLAUDE BERNARD LYON 1, Villeurbanne (FR)

(21) Appl. No.: 18/693,112

(22) PCT Filed: Sep. 20, 2022
(86) PCT No.: PCT/EP2022/076100

§ 371 Date: Oct. 10, 2024

(30) Foreign Application Priority Data
Sep. 20, 2021 (FR) FR2109849

(51) Int. Cl. C07K 7/08; A61K 38/00; A61P 17/02; C07K 7/06

(52) U.S. Cl. CPC C07K 7/08; A61P 17/02; C07K 7/06; A61K 38/00

(57) ABSTRACT

The present invention relates to novel peptides having the same motif LRIRX1TYX2, where X1 is selected from amino acids A or F; and X2 is selected from amino acids G or K. The present invention also relates to their use in the treatment of skin changes of various origins, to pharmaceutical and cosmetic compositions comprising them, and to a cosmetic method. More particularly, the treatment of said changes consists in reinforcing the dermal-epidermal junction, cell-matrix and/or cell-cell adhesion and cell migration in the epidermis, and in promoting the repair of the skin surface.

### CROSS-REFERENCE TO RELATED PATENT APPLICATIONS

This application is a U.S. National Stage Application under 35 U.S.C. § 371 of International Patent Application No. PCT/EP2022/076100, filed Sep. 20, 2022, which claims priority to French Patent Application No. 2109849, filed Sep. 20, 2021, both of which are incorporated by reference in their entireties. The International Application was published on Mar. 23, 2023, as International Publication No. WO/2023/041801 A1.

### SEQUENCE LISTING

A sequence listing, prepared on Oct. 9, 2024 as the ST26 text file “LAJ B004 PCT US1-SEQUENCE LISTING VIPO V2 FULL.xml” having a file size of 24,458 bytes, is incorporated herein by reference in its entirety.

### FIELD OF THE INVENTION

The present invention relates to novel peptides having the same motif LRIRX₁TYX₂ (SEQ ID NO: 20), where X₁ is selected from amino acids A or F; and X₂ is selected from amino acids G or K. The present invention also relates to their use in the treatment of skin changes of various origins, to pharmaceutical and cosmetic compositions comprising them, and to a cosmetic method. More particularly, the treatment of said changes consists in reinforcing the dermal-epidermal junction, cell-matrix and/or cell-cell adhesion and cell migration in the epidermis, and in promoting the repair of the skin surface.

### PRIOR ART

The most common and natural alteration of the skin is quite simply the kind caused by skin aging. Skin aging is a complex phenomenon that is due to numerous intrinsic factors (genetic factors) and extrinsic factors (such as the sun, diet, exposure to cigarette smoke, etc.). Clinically, the appearance of wrinkles and fine lines, a loss of cutaneous elasticity, a loosening of cutaneous and subcutaneous tissues, and poorer healing are all observed.

Many research pathways have been proposed to combat skin aging, including protection against the environment (sun, pollution, etc.), activation of cell regeneration, reinforcement of the extracellular matrix (collagen and elastin). Recently, studies have shown the importance of the adhesion of the cells to one another on the one hand, and of the adhesion between the cells and the extracellular matrix on the other hand, in the skin aging process and therefore of the need to reinforce them to prevent or even treat skin loosening.

The dermatological studies recently looking into the use of peptides in cutaneous biology (sequences derived from alpha-MSH, certain neuropeptides, RGD peptide, etc.), are geared towards the search for peptides having a strong activity at the cutaneous level.

The cosmetic industry is also constantly expecting new peptides capable of increasing the adhesion of the cells to the extracellular matrix and/or the adhesion of the cells to one another.

The interactions between the cells and the extracellular matrix (ECM) indeed play an important role in controlling cell behavior. This control is carried out by specific interactions between the matrix molecules and the cells at transmembrane receptors, mainly from the integrin family, whose intracellular domain is linked to the constituents of the cytoskeleton. This allows the matrix to ensure the transmission of intracellular signals that modulate, depending on the case, the adhesion, migration, proliferation, differentiation or apoptosis of the cells of the epidermis. This control of the cellular behavior is crucial during development but also during tissue renewal.

Depending on the constituent molecules and the organization that results therefrom, several types of ECM are distinguished, the basal lamina undoubtedly being the most specialized. The basal lamina are continuous protein matrix ends on which the various sheets of the organism's cells rest. They have long been defined as discrete morphological structures whose function was limited to partitioning the tissue compartments. It is only over the last 25 years that, despite their low representativeness and their high insolubility, significant advances have been carried out in the investigation of their molecular composition and their functions. It appears that these structures play a fundamental role in controlling cell behavior, both during embryonic development and in maintaining the integrity of differentiated cell phenotypes.

The structure of the skin is composed of:
a covering epithelium: the epidermis,
connective tissue: the dermis (thick layer determining the morphology of the skin and containing the blood and lymphatic vessels, the nerves), and
adipose tissue; the hypodermis.

The dermis and epidermis are linked by a single and complex structure, the dermal-epidermal junction (DEJ) or epidermal basal lamina. Anatomically, it corresponds to the zone between the basal cells of the epidermis and the most superficial layers of the dermis. It is an adhesion zone that determines partitioning between the polarized epithelium and the underlying stroma, ensuring the maintenance and cohesion of the adjacent cells. The DEJ, composed exclusively of ECMs, carries out two roles:
1°) a mechanical role, its unique molecular architecture gives it high mechanical stability, allowing it to ensure the solid anchoring of the epidermis;
2°) a biological role, the proteins of the DEJ maintain narrow relationships with the basal cells of the epidermis and transmit to them significant morphogenetic information via receptors of the integrin family.

The DEJ is also a scattering filter with respect to nutrient and metabolic elements. It therefore enables the transmission of the biological information.

During skin aging, the DEJ is flattened and gradually loses its characteristic undulations, which considerably reduces the epidermis-dermis interface. In addition, the molecular networks become disorganized, the protein structure is weakened, and the adhesion of the basal keratinocytes is reduced.

Consequently, the mechanical function (support) and the biological function (exchange of information and molecules) of the DEJ are altered.

When there is a skin injury, the DEJ is damaged and its constituent molecules are degraded by specific enzymes. Under favorable conditions, epidermal re-epithelialization begins a few hours after trauma. As soon as the epithelium has covered the bed of the wound, the proteins of the DEJ reappear, sequentially and ordered.

With skin aging, each of these steps takes place more slowly. In particular, a decrease in the expression of the matrix adhesion proteins as well as that of the membrane receptors was observed, which could constitute the major cause of the delay observed in the DEJ reconstitution process and of the extreme fragility of the healing tissues in elderly subjects.

Laminin 5 (LN-5) or laminin 332 is a protein specifically expressed in basal lamina of the squamous and transitional epithelia that are specialized as the skin's DEJ. LN-5 results from the heterotrimeric assembly of the alpha 3, beta 3 and gamma 2 subunits and is synthesized exclusively by epithelial cells in the form of a 460 kDa precursor. Under physiological conditions, each of the alpha 3 and gamma 2 subunits undergoes an extracellular post-translational cleavage of the carboxy- and amino-terminal ends respectively, resulting in the mature tissue form. Studies indicate that the entirety of the gamma 2 precursor is necessary for the secretion and the integration of the LN-5 into the ECM. Other studies have shown that the gamma 2 precursor chain is involved in the migration of keratinocytes during the skin healing process.

The major role of LN-5 is underlined by the existence of hereditary or acquired pathologies, resulting from anomalous synthesis and/or expression of its constituent subunits. These diseases, called junctional epidermolysis bullosa, lead in particular to a fragility of the skin of the skin characterized by the spontaneous formation of epidermal blisters. LN-5 thus plays a crucial and irreplaceable role in epithelial-mesenchymal cohesion. LN-5 carries decisive biological signals since it allows the adhesion of the adjacent epithelial cells via integrins and induces the assembly of the stable adhesion structures that are hemidesmosomes.

International patent application WO 00/66731 to Biostatum describes the production of whole LN-5 in recombinant form in eukaryotic cells (L5r) and documents its activity at improving healing, growth, and cellular adhesion. Other peptides derived from Laminin-5 are described in U.S. Pat. No. 6,294,356 (Jones).

Patent EP1784423, filed by the Applicant, characterizes the efficacy of a peptide with the sequence TALRIRATYGEY (SEQ ID NO: 17), a sequence present on gamma chain 2 of LN-5, which specifically induces the adhesion of the keratinocytes of the epidermis and other epithelial cells.

There remains a need for alternatives for the treatment of skin alterations of various origins, as described above, which additionally is more efficient.

Today, the Applicant has identified, surprisingly and unexpectedly, that an effective amount of a peptide comprising the motif LRIRX₁TYX₂ (SEQ ID NO: 20), where X₁ is selected from amino acids A or F and X₂ is selected from amino acids G or K, specifically induces the adhesion of the keratinocytes of the epidermis and other epithelial cells. This peptide not only makes it possible to increase the adhesion of the cells to the ECM, but also to increase the migration of keratinocytes. Due to its small size and its great stability, the peptide has all the characteristics required to suitably pass through the epidermis and reach its target, the DEJ, and interact with the basal keratinocytes and transmit adhesion induction signals. Its method of manufacturing by chemical synthesis is simple and applicable on an industrial scale. It does not require the use of cell cultures of animal origin, nor to growth factors and/or serums or derivatives of serums. Being of small size, it will not be, or will rarely be, the target of specific and/or non-specific proteolytic degradations.

The general inventive concept according to the invention is the use of a peptide comprising the sequence: LRIRX₁TYX₂ (SEQ ID NO: 20), where:
X₁ is selected from amino acids A or F; and
X₂ is selected from amino acids G or K.

It is this motif that specifically induces the effects mentioned below. The variants making it possible to cyclize or double the presence of this peptide are only variant formats of this motif that is the object of the invention, which can slightly influence its effectiveness.

### DISCLOSURE OF THE INVENTION

According to a first aspect, the invention relates to a peptide consisting of the sequence: LRIRX₁TYX₂ (SEQ ID NO: 20), where:
X₁ is selected from amino acids A or F; and
X₂ is selected from amino acids G or K.

According to one embodiment, said peptide has the following sequence: LRIRFTYK (SEQ ID NO: 1)

According to one embodiment, said peptide has the following sequence: LRIRFTYG (SEQ ID NO: 5)

According to one embodiment, said peptide has the following sequence: LRIRATYK (SEQ ID NO: 9)

According to one embodiment, said peptide has the following sequence: LRIRATYG (SEQ ID NO: 13)

According to a second aspect, the invention relates to a peptide consisting of the sequence: CLRIRX₁TYX₂C (SEQ ID NO: 21), where:
X₁ is selected from amino acids A or F; and
X₂ is selected from amino acids G or K.

The addition of these two cysteines to the ends of the peptide allows its cyclization.

According to one embodiment, said peptide has the following sequence: CLRIRFTYKC (SEQ ID NO: 2)

According to one embodiment, said peptide has the following sequence: CLRIRFTYGC (SEQ ID NO: 6)

According to one embodiment, said peptide has the following sequence: CLRIRATYKC (SEQ ID NO: 10)

According to one embodiment, said peptide has the following sequence: CLRIRATYGC (SEQ ID NO: 14)

According to a third aspect, the invention relates to a peptide consisting of the sequence: CSGLRIRX₁TYX₂SGC (SEQ ID NO: 22), where:
X₁ is selected from amino acids A or F; and
X₂ is selected from amino acids G or K.

The addition of these two cysteines as well as serine-glycine spacers at the ends of the peptide allows its cyclization.

According to one embodiment, said peptide has the following sequence:

(SEQ ID NO: 3)
CSGLRIRFTYKSGC

According to one embodiment, said peptide has the following sequence:

(SEQ ID NO: 7)
CSGLRIRFTYGSGC

According to one embodiment, said peptide has the following sequence:

(SEQ ID NO: 11)
CSGLRIRATYKSGC

According to one embodiment, said peptide has the following sequence:

(SEQ ID NO: 15)
CSGLRIRATYGSGC

According to a fourth aspect, the invention relates to a peptide consisting of the sequence LRIRX₁TYX₂SGLRIRX₁TYX₂ (SEQ ID NO: 23), where:
X₁ is selected from amino acids A or F; and
X₂ is selected from amino acids G or K.

The addition of these serine-glycine spacers makes it possible to space this peptide, which twice comprises the motif of the peptide according to the invention.

According to one embodiment, said peptide has the following sequence:

(SEQ ID NO: 4)
LRIRFTYKSGLRIRFTYK

According to one embodiment, said peptide has the following sequence:

(SEQ ID NO: 8)
CSGLRIRFTYGSGCLRIRFTYGSGLRIRFTYG

According to one embodiment, said peptide has the following sequence:

(SEQ ID NO: 12)
CSGLRIRATYKSGCLRIRATYKSGLRIRATYK

According to one embodiment, said peptide has the following sequence:

(SEQ ID NO: 16)
LRIRATYGSGLRIRATYG

The present invention also relates to other peptides resulting from one or more modification(s) of the aforementioned peptides, the elimination or substitution of one or more amino acids, preferably as indicated in table 1, and/or of an oligomerization, cyclization, or folding of the peptides above, it being understood that these modifications do not in any way reduce the adhesive activity of the reference peptide, or may even improve it.

TABLE 1
Possible substitutions of the amino acids
of the peptides according to the invention

| Positions of | | Possible replacement |
|---|---|---|
| amino acids | SEQ | amino acids |
| 1 | L | L, M, I, V, F, E or R |
| 2 | R | R, N, E, Q, H, K or Y |
| 3 | I | I, M, L, V, F, T or K |
| 4 | R | R, N, E, Q, H, K or T |
| 5 | X (A or F) | A, C, S, G, V or D |
| 6 | T | T, S, P, G, N, D, E, Q, H, or K |
| 7 | Y | Y, H, F, W, D, I or Q |
| 8 | X (G or K) | G, S, T, A or N |

* X may be any amino acid.

More particularly, the addition or removal of one or more amino acids can be carried out on the carboxy side and/or on the amino-terminal side of said peptide. The invention also relates to any analog or derivative that would result from the transplantation of a motif of interest (natural or synthesis molecules, proteins and/or sugars) on said peptide. It also relates to any dermatologically active moiety of the peptide of the invention, modified or not.

Preferably, the peptides according to the invention are obtained by chemical synthesis.

The invention also relates to the use of said peptides according to the invention as a medication.

According to one embodiment, said peptides according to the invention are used in the reinforcement of the dermal-epidermal junction, cell-cell adhesion and/or cell-matrix adhesion and cell migration at the epidermis.

As indicated above, the alteration of the epidermis and the DEJ most commonly observed is that resulting from skin aging. However, other skin alterations may occur independently of aging and may for example be induced by certain dermatological pathologies. These may include eczema, psoriasis, pruritus, irritant dermatitis, helioderma, keratoses, mycoses, ichthyosis. In addition to the symptoms specific to each of these pathologies, the skin undergoes alterations, which the present invention proposes to remedy by way of therapeutic supplements. It is clear that the present invention is not intended to treat such pathologies, but rather to restore the damaged DEJ and cell adhesion in these pathologies and regeneration of the epidermis by promoting the migration of keratinocytes.

Thus, the present invention allows the repair, regeneration and/or restructuring of the skin.

The skin may also be weakened by cosmetic or therapeutic treatments of the skin, which, while treating the targeted pathology, generate side effects on the skin that should be treated independently of the pathology itself. This is the case in particular for acne treatments, PUVA treatments, surgical procedures (dermatological or otherwise), laser skin treatments, dermabrasion, skin peelings, or treatment of cancers by radiotherapy.

According to one embodiment, said peptides according to the invention are used in the treatment of skin alterations induced by dermatological pathologies.

According to one embodiment, said peptides according to the invention are used in treating impairments of skin weakened by cosmetic or therapeutic treatments.

According to one embodiment, said peptides according to the invention are used in the curative or preventive treatment of skin aging such as wrinkles, loosening, loss of elasticity, reduced healing, cheloid scars, hypertrophic or atrophic, senile xerosis, modifications of the pigment system, depletion of the vascular network of the skin, irregularity of the skin grain, skin atrophy and alteration of the skin appendages.

Indeed, the abovementioned pathologies, skin weakening and treatments cause alterations to the skin, such as reducing the adhesion of the epidermis and epidermal cohesion, or even worse restructuring of the skin surface.

According to another aspect, the invention relates to a pharmaceutical composition comprising at least one peptide according to the invention.

Preferably, said pharmaceutical composition according to the invention comprises from 0.00002% to 5%, preferably from 0.00005% to 0.1% and even more preferentially from 0.0001% to 0.001% by weight of the peptide according to the invention and at least one pharmaceutically acceptable excipient.

According to one embodiment, said composition further comprises at least one other dermatologically active ingredient.

More particularly, the composition according to the present invention further comprises at least one other dermatologically active ingredient acting either on the reinforcement of the dermal-epidermal junction, the cell-matrix adhesion of the skin, and/or the cell-cell adhesion and/or the migration of keratinocytes, or otherwise on the skin depending on the nature of the agent(s) used, such as a moisturizing agent, a lipid-restoring agent, a superfatting agent, an exfoliating agent, a keratolytic agent, an antioxidant agent, a soothing agent, a softening agent, a sedative agent, a cleaning agent, a makeup-removing agent, a sanitizing agent, an antibacterial agent, an antiseptic agent, an antiseborrheic agent, a lightening agent, an anti-wrinkle agent, a decongestant agent, a revitalizing agent, a cell renewal activating agent or one or more solar filters.

The compositions of the invention must be in a dermatologically acceptable form, that is to say compatible with the skin, body hair, mucous membranes and/or head hair.

Thus, preferably, said pharmaceutical composition according to the invention is in the form of a cream, ointment, ointment, mask, serum, milk, lotion, paste, foam, aerosol, stick, powder, solution, suspension, shampoo, conditioner, patch, aqueous or oily hydro-alcoholic solution, oil-in-water or water-in-oil emulsion or multiple emulsions, aqueous or oily gel, suture, surgical glue, dressing, a liquid, pasty or solid anhydrous product, and/or a dispersion of oil in an aqueous phase using spherules, these spherules being polymeric nanoparticles such as nanospheres and nanocapsules or lipid vesicles of ionic and/or non-ionic type.

Preferably, this composition is intended for topical application.

Any additional compounds, active or not, may be added to the composition of the invention, and a person skilled in the art will choose them so that their addition does not alter the advantageous properties of said composition.

According to another aspect, the invention relates to a non-therapeutic cosmetic composition comprising at least one peptide according to the invention.

Preferably, said cosmetic composition according to the invention comprises from 0.00002% to 5%, preferably from 0.00005% to 0.1% and even more preferentially from 0.0001% to 0.001% by weight of the peptide and at least one cosmetically acceptable excipient.

According to one embodiment, said composition further comprises at least one other cosmetically active ingredient.

The composition of the invention may also contain the usual adjuvants in cosmetic and dermatological fields, such as hydrophilic or lipophilic gelling agents, hydrophilic or lipophilic active agents, preservatives, antioxidants, solvents, fragrances, fillers, filters, pigments, odor absorbers, and dyes. The amounts of these various adjuvants are those conventionally used in the fields under consideration, and for example from 0.01 to 20% of the total weight of the composition. These adjuvants, depending on their nature, can be introduced into the fatty phase, into the aqueous phase, into the lipid vesicles and/or into the nanoparticles.

Preferably, said composition is in the form of a cream, ointment, ointment, mask, serum, milk, lotion, paste, foam, aerosol, stick, powder, solution, suspension, shampoo, conditioner, patch, aqueous or oily hydro-alcoholic solution, oil-in-water or water-in-oil emulsion or multiple emulsions, aqueous or oily gel, a liquid, pasty or solid anhydrous product, and/or a dispersion of oil in an aqueous phase using spherules, these spherules being polymeric nanoparticles such as nanospheres and nanocapsules or lipid vesicles of ionic and/or non-ionic type.

According to one embodiment, said composition is a cosmetic composition for caring for skin aging such as wrinkles, loosening, and loss of elasticity.

According to one embodiment, said composition is a cosmetic composition for caring for the regeneration or repair of the skin.

According to one embodiment, said composition is a cosmetic composition for caring for the scalp such as hair loss care.

According to one embodiment, said composition is a cosmetic composition for caring for the pigment system of the skin, the irregularity of the skin grain, and/or the alteration of the skin appendages.

According to another aspect, the invention relates to the non-therapeutic use of at least one peptide according to the invention in a cosmetic composition.

According to a final aspect, the invention relates to a method for cosmetic care of the skin characterized in that it comprises a step of applying to the skin a cosmetic composition according to the invention.

Preferably, an amount of 0.2 to 3 mg/cm2 of cosmetic composition is applied to the area of the skin, preferably 2 mg/cm2.

Preferably, the cosmetic composition according to the invention is applied once to twice daily, preferably twice daily, for at least 7 days, preferably at least 15 days, even more preferably at least one month and particularly preferably at least 2 months. Most particularly preferably, the cosmetic composition is applied in the method according to the invention twice per day for at least 2 months.

### DESCRIPTION OF THE FIGURES

Other features, aims and advantages of the invention will become apparent from the following description, which is purely illustrative and non-limiting, and which must be read with reference to the attached drawings wherein:

FIG. 1 shows the results of the cell adhesion test for the TALRIRATYGEY peptide (SEQ ID NO: 17) as positive control and peptides LRIRATYG (SEQ ID NO: 13), LRIRATYGLRIRATYG (SEQ ID NO: 18) and LRIRATYGSGLRIRATYG (SEQ ID NO: 16).

FIG. 2 shows the results of the cell adhesion test for the TALRIRATYGEY peptide (SEQ ID NO: 17) as positive control and peptides LRIRATYG (SEQ ID NO: 13), LRIRATYGLRIRATYG (SEQ ID NO: 18), CSGLRIRATYGSGC (SEQ ID NO: 15) and LRIRATYGSGLRIRATYG (SEQ ID NO: 16).

FIG. 3 shows the results of the wound closure test of a sheet of primary keratinocytes for the peptide TALRIRATYGEY (SEQ ID NO: 17), the peptides LRIRATYG (SEQ ID NO: 13), LRIRATYGLRIRATYG (SEQ ID NO: 18), the cyclic peptide CSGLRIRATYGSGC (SEQ ID NO: 15), a positive control (EGF) and a negative control (culture medium alone). (The *s correspond to: * p<0.05; ** p<0.005; ****p<0.0001).

FIG. 4 shows the results of a second wound closure test of a sheet of primary keratinocytes for the peptide TALRIRATYGEY (SEQ ID NO: 17), the peptide LRIRATYG (SEQ ID NO: 13), the cyclic peptide CSGLRIRATYGSGC (SEQ ID NO: 15), a positive control (EGF) and a negative control (culture medium alone). (The *s correspond to: * p<0.05; ** p<0.005; ****p<0.0001).

FIG. 5 shows the results of the cell adhesion test for the TALRIRATYGEY peptide (SEQ ID NO: 17) as positive control and peptides LRIRATYG (SEQ ID NO: 13), LRIRATYK (SEQ ID NO: 9), and LRIRKTYG (SEQ ID NO: 19).

FIG. 6 shows the results of the cell adhesion test for the TALRIRATYGEY peptide (SEQ ID NO: 17) and the peptides LRIRATYG (SEQ ID NO: 13), LRIRFTYK (SEQ ID NO: 1), and LRIRFTYG (SEQ ID NO: 5).

FIG. 7 shows the results of the wound closure test of a sheet of primary keratinocytes for the peptide TALRIRATYGEY (SEQ ID NO: 17) and the peptides LRIRATYG (SEQIDNO: 13), LRIRFTYG (SEQIDNO: 5) and LRIRFTYK (SEQIDNO: 1), a positive control (EGF) and a negative control (culture medium alone). (The *s correspond to: * p<0.05; ** p<0.005; ** p<0.0005; **** p<0.0001).

FIG. 8 shows the results of the cell adhesion test for the peptide LRIRFTYK (SEQ ID NO: 1), and the cyclic peptides CLRIRFTYKC (SEQ ID NO: 2) and CSGLRIRFTYKSGC (SEQ ID NO: 3).

FIG. 9 shows the results of the cell viability test for the peptides according to the invention CSGLRIRFTYKSGC (SEQ ID NO: 17), LRIRATYG (SEQ ID NO: 13), LRIRFTYK (SEQ ID NO: 1), as well as for the cyclic peptide CLRIRFTYKC (SEQ ID NO: 2).

FIG. 10 shows the results of the wound closure test of a sheet of primary keratinocytes for the cyclic peptides according to the invention CLRIRFTYKC (SEQ ID NO: 2), CSGLRIRFTYKSGC (SEQ ID NO: 3) and for the peptides LRIRFTYK (SEQ ID NO: 1), LRIRATYG (SEQ ID NO: 13), TALRIRATYGEY (SEQ ID NO: 17), as well as for EGF, and negative control. (The *s correspond to: * p<0.05; ** p<0.0005; ****p<0.0001)

### EXAMPLES

### Example 1: Equipment and Method

### The Cells Used for the Study:

the HT1080 line: Cells originating from epithelial lines, tools commonly used for cell adhesion studies, were first used. The cells of the HT1080 line (fibrosarcoma, Human), American Type Culture Collection CCL-121.
primary human keratinocytes: For the cell viability and migration test, freshly isolated normal human keratinocytes were used. Human keratinocytes were obtained from preputial biopsy (surgical waste, tissue bank and cells, Edouard Herriot Hospital). The culture medium used during the experiments was the medium defined for KGM-Gold keratinocytes sold by Lonza Bioscience containing 0.15 mM of CaCl2, pH 7.2 to 7.4. The keratinocytes were obtained according to the Boyce and Ham technique (Cultivation, frozen storage, and clonal growth of normal human epidermal keratinocytes in serum free-media, Tiss Cuit. Meth. 1985, 9:83-93). The pieces of skin, after rinsing in PBS buffer containing antibiotics, were freed of the fat tissue and cut into 3 mm² pieces, then placed in a sterile solution of 0.25% trypsin in PBS for 16 h at 4° C. The dermis/epidermis separation was carried out in a Petri dish containing culture medium in order to stop the action of trypsin. The epidermis fragments were sucked in and discharged several times to detach the free basal cells. After centrifuging, 3.10⁴ live cells were seeded per cm2 on dishes for tissue cultivation (Corning, Polylabo, France). The keratinocytes were cultured at 37° C. in a CO2 incubator (5% CO2, 95% air and 98% moisture). The sub-culture took place when the cells reached the sub-confluence. The sheet of cells was then rinsed with PBS, then covered with a solution of Trypsin-EDTA (0.05-0.02%). After a short incubation at 37° C., the cells were detached, counted, and re-seeded.

### Cell Viability Test:

The effect of the peptides on cell viability was analyzed using a colorimetric test (Cell Proliferation KitXTT, Roche Diagnostics, Meylan, France) on normal human keratinocytes from young subjects.

The chemical reaction of the test is based on the NADPH production of the living cells allowing the reduction of yellow tetrazolium XTT salts into orange formazan salts. The absorbance measurement is performed at 490 nm using an ELISA microplate reader. The cells were seeded in 96-well plates at a rate of 10⁴ cells per well (3/condition) in KBM-Gold culture medium. After 24 h of culture at 37° C. in the presence of 5% CO2, the culture media were removed and replaced with KBM-Gold medium containing the quantities of peptide indicated in the figures. Two days later, the media were then removed and replaced by the test reagent. The plates were then placed in an incubator at 37° C. and the absorbance was read at 5 h.

Controls without peptide were carried out on the same plate. The results are presented in the form of the percentage of the viability of the cells brought into the presence of the peptide relative to the controls without peptide. The cell viability was calculated according to the formula:

$$\% viability = ( Abs . cells with peptide / Abs . control cells ) \times 100.$$

Quantitative analysis of the cellular adhesion properties of the peptides of interest by a colorimetric test:

### Preparation of the Adhesion Substrates

A decreasing concentration range of the peptides was carried out by successive dilution in PBS (Phosphate Buffer Saline, KH2PO4 1.54 mM; Na2HPO4 1.42 mM; 131 mM NaCl), from a starting solution at 1 mg/ml. These solutions were immediately distributed on 96-well culture plates (plates chosen according to the immobilization) at a rate of 100 μl per well. The plates were then placed at +4° C. for 16 to 18 h. The solutions were then removed and each well was saturated with a solution of PBS-BSA 1% (bovine serum albumin). Three additional wells without substrate underwent the same treatment and served as a negative control.

### Cell Adhesion Test

The HT1080 cells were detached from the culture dishes by a trypsin/EDTA solution (0.05-0.02%, then were suspended in DMEM medium without additives. The number of cells seeded is 100,000 cells per well.

### Assessment of the Cell Adhesion Test

After seeding the cells, the plates were placed in an incubator at 37° C. in a 5% CO2 atmosphere. After incubation for 30 to 60 minutes, the adhesion medium was removed and each well was washed with a solution of sterile PBS in order to remove the cells that did not adhere. The remaining cells, adhering to the substrate, were fixed by a 1% glutaraldehyde solution in PBS, for 15 minutes. The glutaraldehyde solution was removed and the cells were stained with a crystal violet solution diluted to 1% in distilled water for 30 minutes. After significant rinsing, the cells were permeabilized with a triton solution at 0.02% for 15 minutes, in order to solubilize the crystal violet. The absorbance was read at 570 nm using a plate reader (Victor² V Spectrophotometer, Perkin Elmer). Each experimental point was done in triplicate. The negative control value is the average of the absorbance of the three control wells (BSA). This was subtracted from each of the absorbance values obtained for the experimental points. The averages of the absorbance values for each of the conditions were calculated and were presented in the form of a curve, with on the y-axis, the average values of the absorbance, and on the x-axis, the various quantities of substrate immobilized in the wells (in μg or mole).

### Optimization and Verification of the Immobilization of the Peptides in the Plates

The various peptides tested do not necessarily have the same fillers; they are differently immobilized on the plates, which can lead to erroneous results. Thus, it is necessary to test various types of plates and different immobilization buffers in order to ensure good immobilization of the peptides, especially in comparative studies.

Thus, the series of peptides were immobilized under the conditions described in the adhesion test, and after the immobilization, the amount of peptide immobilized is assayed by a colorimetric assay method.

This assay is carried out using the micro-BCA colorimetric assay kit (PIERCE UPTIMA distributed by Interchim) wherein the intensity of the color is directly proportional to the content of peptide bonds that have reduced the Cu2+ ions into Cu+ ions, during a reaction which is concentration-dependent. Bicinchoninic acid is a specific chromogenic reagent for copper (I) forming therewith a purple complex having a maximum absorbance at 562 nm which is directly proportionate to protein concentrate. The proteins are assayed in each well using the BCA assay technique; 25 μL of PBS are added to the wells and are incubated for 30 minutes at 37° C. in the presence of 200 μL of BCA reagent prepared extemporaneously according to the protocol provided in the kit. The OD is then read at 562 nm (Victor² V Spectrophotometer) and the protein content is determined relative to a standard range produced from a commercial protein standard of BSA at 2 mg/mL.

### Wound Closure Test of a Sheet of Primary Keratinocytes

6-well plates (Costar) are covered with collagen I for 16 h then rinsed with PBS. Three inserts with two Ibidi-sterile wells (Ibidi, reference: 80209) are deposited in each of the wells. 2×10⁴ keratinocytes are deposited in each insert well in KBM-Gold. After two hours of incubation in humid air at 37° C., composed of 5% CO2 and 95% atmospheric air, the culture medium is sucked out and the inserts removed. KBM-Gold with no supplement is added to each of the wells of the plate. The cells are thus: not treated (medium alone), treated with supplement-free KBM-Gold containing the peptides studied (25 μg/ml), or treated with supplement-free KBM-Gold containing EGF at 10 ng/ml (Peprotech). The cultures are stopped at 12 h and 24 h, the media are sucked out, and the cells are fixed with 2% PBS-Paraformaldehyde for 20 minutes. After the fixator has been sucked out, the cells are colored with a 0.1% solution of Crystal Violet in ultrapure water for 30 minutes. The Crystal Violet is removed and the wells are rinsed with water. Image acquisitions are carried out and the injuries are quantified using Adobe Photoshop CS3 software.

### Example 2: Adhesion Test-Comparison Between the Peptide TALRIRATYGEY (SEQ ID NO: 17) and the Peptides LRIRATYG (SEQ ID NO: 13), LRIRATYGLRIRATYG (SEQ ID NO: 18) and LRIRATYGSGLRIRATYG (SEQ ID NO: 16)

The adhesion test was carried out according to the protocol described above with HT1080 cells. The peptide TALRIRATYGEY (SEQ ID NO: 17) was used as a control peptide, for testing the peptides:

(SEQ ID NO: 13)
LRIRATYG
(SEQ ID NO: 18)
LRIRATYGLRIRATYG
(SEQ ID NO: 16)
LRIRATYGSGLRIRATYG

A protein assay was carried out to determine the amount of peptide immobilized in each well in order to adjust it if necessary.

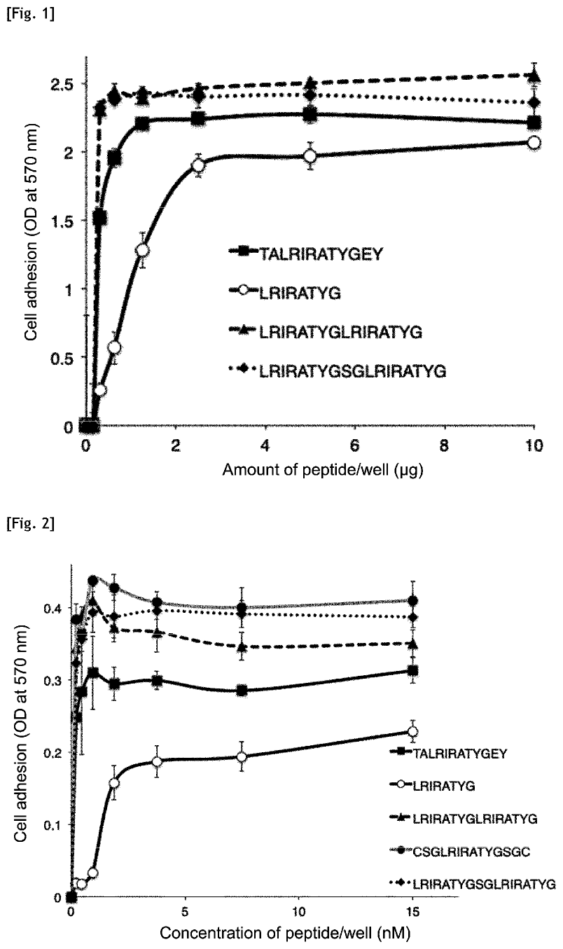

The results are presented in FIG. 1.

These results show that the duplication of the LRIRATYG motif (SEQ ID NO: 13) leads to a doubling of low-concentration adhesion induction activity.

### Example 3: Adhesion Test-Comparison Between the Peptide TALRIRATYGEY (SEQ ID NO: 17), the Peptides LRIRATYG (SEQ ID NO: 13), LRIRATYGLRIRATYG (SEQ ID NO: 18), LRIRATYGSGLRIRATYG (SEQ ID NO: 16) and the Cyclic Peptide CSGLRIRATYGSGC (SEQ ID NO: 15)

The adhesion test was carried out according to the protocol described above with HT1080 cells. The peptide TALRIRATYGEY (SEQ ID NO: 17) was used as a control peptide, for testing the peptides:

(SEQ ID NO: 13)
LRIRATYG
(SEQ ID NO: 18)
LRIRATYGLRIRATYG
(SEQ ID NO: 15)
CSGLRIRATYGSGC
(SEQ ID NO: 16)
LRIRATYGSGLRIRATYG

A protein assay was carried out to determine the amount of peptide immobilized in each well in order to adjust it if necessary.

The results are presented in FIG. 2.

These results particularly show that the cyclization of the LRIRATYG motif (SEQ ID NO: 13), giving the peptide CSGLRIRATYGSGC (SEQ ID NO: 15) leads to the activity being doubled at a low concentration.

### Example 4: Wound Closure Test of a Sheet of Freshly Isolated Primary Keratinocytes, with the Peptides TALRIRATYGEY (SEQ ID NO: 17), LRIRATYG (SEQ ID NO: 13), LRIRATYGLRIRATYG (SEQ ID NO: 18), and the Cyclic Peptide CSGLRIRATYGSGC (SEQ ID NO: 15)

The wound closure test was carried out according to the protocol described above (Example 1) with freshly isolated human primary keratinocytes. The recombinant human Epidermal Growth Factor (EGF) (Peprotech, France) was used as a positive control, and the culture medium alone as a negative control.

The peptides tested are:

(SEQ ID NO: 17)
TALRIRATYGEY
(SEQ ID NO: 13)
LRIRATYG
(SEQ ID NO: 18)
LRIRATYGLRIRATYG
(SEQ ID NO: 15)
CSGLRIRATYGSGC

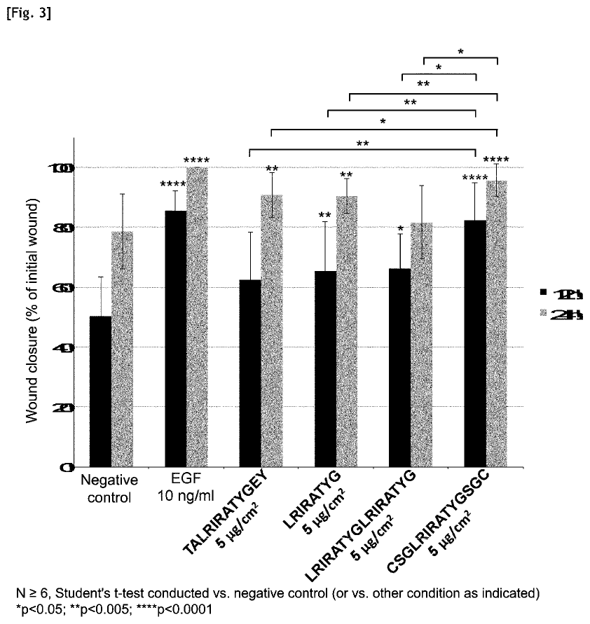

The results are presented in FIG. 3.

These results show that all the peptides studied significantly promote wound closure within 12 h and/or 24 h post-injury in comparison with the negative control. The cyclic peptide CSGLRIRATYGSGC (SEQ ID NO: 15) has an activity significantly greater than that of the other peptides and very significantly greater than that of the negative control both of the times it was tested.

### Example 5: Wound Closure Test of a Sheet of Freshly Isolated Primary Keratinocytes, with the Peptides TALRIRATYGEY (SEQ ID NO: 17), LRIRATYG (SEQ ID NO: 13), and the Cyclic Peptide CSGLRIRATYGSGC (SEQ ID NO: 15)

The wound closure test was carried out according to the protocol described above (Example 1) with freshly isolated human primary keratinocytes. The recombinant human Epidermal Growth Factor (EGF) (Peprotech, France) was used as a positive control, and the culture medium alone as a negative control.

The peptides tested are:

(SEQ ID NO: 17)
TALRIRATYGEY
(SEQ ID NO: 13)
LRIRATYG
(SEQ ID NO: 15)
CSGLRIRATYGSGC

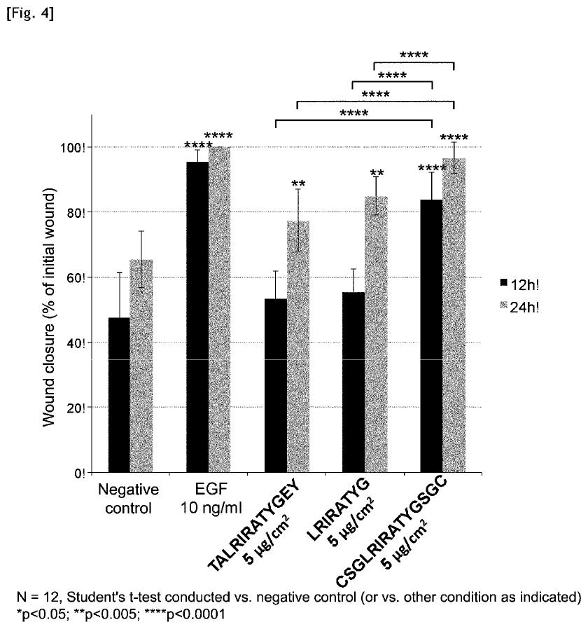

The results are presented in FIG. 4.

These results show that the peptides studied, in particular the cyclic peptide CSGLRIRATYGSGC, has a significantly greater activity at 12 and 24 h compared to that of the peptides TALRIRATYGEY (SEQ ID NO: 17) and LRIRATYG (SEQ ID NO: 13). In this test, it is also perceived that the peptides LRIRATYG and TALRIRATYGEY have an equivalent activity between them at 12 and 24 h and that the activity at 24 h is significantly greater than that of the negative control. The activity of the cyclic peptide CSGLRIRATYGSGC is significantly greater than that of the negative control.

### Example 6: Adhesion Test-Comparison Between the Peptide TALRIRATYGEY (SEQ ID NO: 17) and the Peptides LRIRATYG (SEQ ID NO: 13), LRIRATYK (SEQ ID NO: 9), LRIRKTYG (SEQ ID NO: 19)

The adhesion test was carried out according to the protocol described above with HT1080 cells. The peptide TALRIRATYGEY (SEQ ID NO: 17) was used as a control peptide, for testing the peptides:

(SEQ ID NO: 13)
LRIRATYG
(SEQ ID NO: 9)
LRIRATYK
(SEQ ID NO: 19)
LRIRKTYG

A protein assay was carried out to determine the amount of peptide immobilized in each well in order to adjust it if necessary.

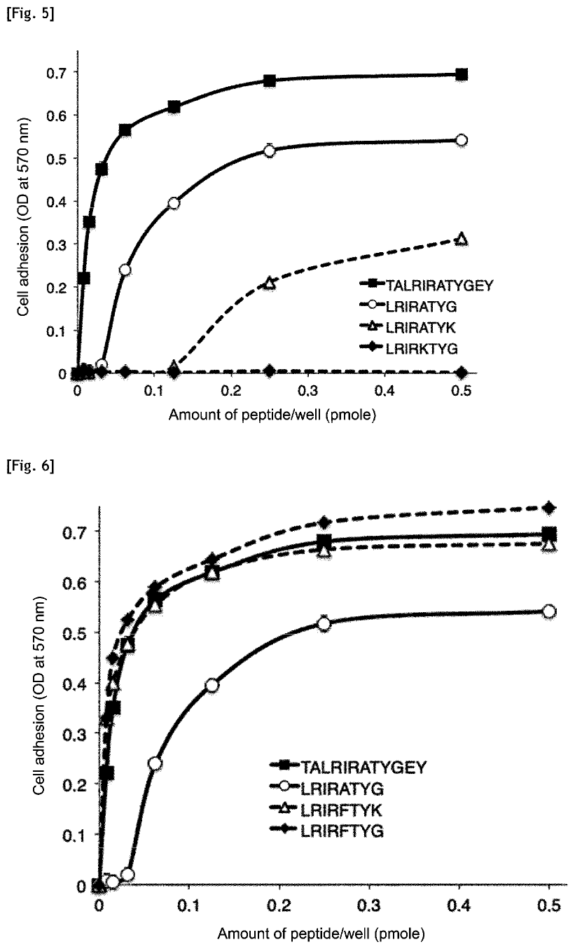

The results are presented in FIG. 5.

These results show that the response dependent on the amount of peptide immobilized in the wells decreases when the K is positioned at the end of the sequence (LRIRATYK SEQ ID NO: 9) and disappears when the K is positioned in the middle of the sequence (LRIRKTYG SEQ ID NO: 19)).

### Example 7: Adhesion Test-Comparison Between the Peptide TALRIRATYGEY (SEQ ID NO: 17) and the Peptides LRIRATYG (SEQ ID NO: 13), LRIRFTYK (SEQ ID NO: 1), LRIRFTYG (SEQ ID NO: 5)

The adhesion test was carried out according to the protocol described above with HT1080 cells.

The peptide TALRIRATYGEY (SEQ ID NO: 17) was used as a control peptide for testing the peptides:

(SEQ ID NO: 13)
LRIRATYG
(SEQ ID NO: 1)
LRIRFTYK
(SEQ ID NO: 5)
LRIRFTYG

A protein assay was carried out to determine the amount of peptide immobilized in each well in order to adjust it if necessary.

The results are presented in FIG. 6.

The response dependent on the amount of peptide immobilized in the wells shows that for the peptides LRIRFTYK (SEQ ID NO: 1), and LRIRFTYG (SEQ ID NO: 5) the adhesion capabilities of the peptide are similar to the TALRIRATYGEY (SEQ ID NO: 17) control.

### Example 8: Wound Closure Test of a Sheet of Freshly Isolated Primary Keratinocytes, with the Peptides TALRIRATYGEY (SEQ ID NO: 17), LRIRATYG (SEQ ID NO: 13), LRIRFTYG (SEQ ID NO: 5) and LRIRFTYK (SEQ ID NO: 1)

The wound closure test was carried out according to the protocol described above (Example 1) with freshly isolated human primary keratinocytes. The peptide TALRIRATYGEY (SEQ ID NO: 17) was used as a control peptide, as well as the recombinant human Epidermal Growth Factor (EGF) (Peprotech, France) as a positive control, and the culture medium alone as a negative control.

The peptides tested are:

(SEQ ID NO: 13)
LRIRATYG
(SEQ ID NO: 1)
LRIRFTYK
(SEQ ID NO: 5)
LRIRFTYG

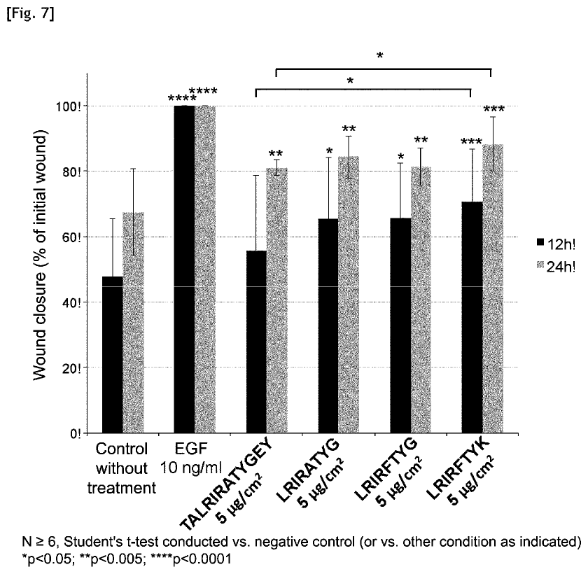

The results are presented in FIG. 7.

These results show that the peptides studied have a significantly greater activity at 12 and 24 h, or even very significantly greater for the peptide LRIRFTYK (SEQ ID NO: 1), compared to the negative control. The peptide LRIRFTYK (SEQ ID NO: 1) has a significantly greater activity than the peptide TALRIRATYGEY (SEQ ID NO: 17) at 12 and 24 h post-wound.

### Example 9: Adhesion Test for Cyclized Peptides (CLRIRFTYKC (SEQ ID NO: 2) and CSGLRIRFTYKSGC (SEQ ID NO: 3) Compared to LRIRFTYK (SEQ ID NO: 1)

The peptides CLRIRFTYKC (SEQ ID NO: 2) and CSGLRIRFTYKSGC (SEQ ID NO: 3) are cyclized versions of the peptide LRIRFTYK (SEQ ID NO: 1). The adhesion test was carried out according to the protocol described above (example 1) with HT1080 cells.

The three peptides were immobilized. Assays were carried out in the wells in order to determine the immobilized amount and to ensure that the same amount of peptide under the different conditions was present, and to adjust it if necessary.

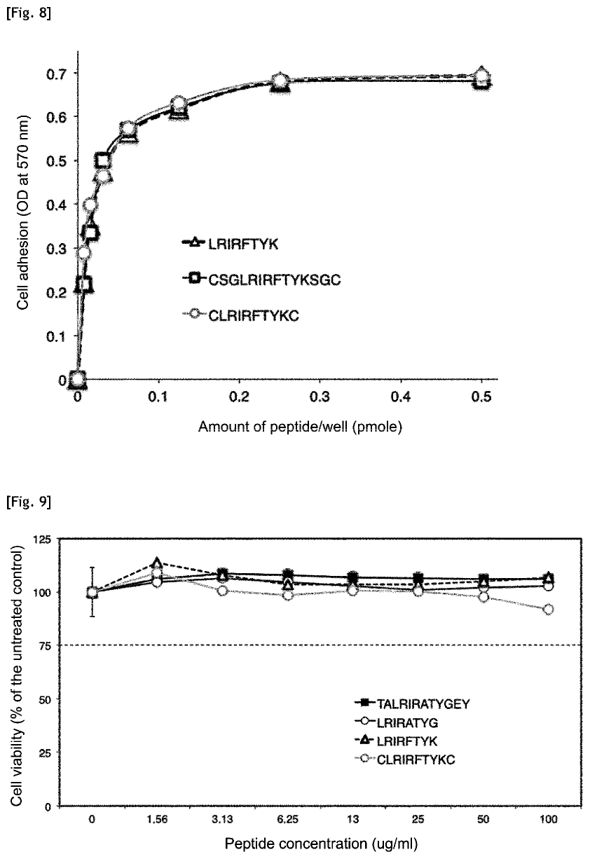

The results are presented in FIG. 8.

The response dependent on the amount of peptide immobilized in the wells shows that all three peptides have an equivalent adhesion activity. Thus, the cyclic without spacers (without SG) is as effective as the cyclic with spacers (SG).

### Example 10: Cell Viability Test on Freshly Isolated Primary Keratinocytes for the Peptides

(SEQ ID NO: 17)
TALRIRATYGEY,
(SEQ ID NO: 13)
LRIRATYG,
(SEQ ID NO: 1)
LRIRFTYK
and
(SEQ ID NO: 2)
CLRIRFTYKC;

The cell viability test was carried out according to the protocol described above (Example 1) with freshly isolated primary keratinocytes. The peptide TALRIRATYGEY (SEQ ID NO: 17) was used as a control peptide.

The peptides tested are:

(SEQ ID NO: 13)
LRIRATYG
(SEQ ID NO: 1)
LRIRFTYK
(SEQ ID NO: 2)
CLRIRFTYKC

The results are presented in FIG. 9.

The results, presented in FIG. 9, show similar positive results for the peptides LRIRATYG (SEQ ID NO: 13), LRIRFTYK (SEQ ID NO: 1), and CLRIRFTYKC (SEQ ID NO: 2) compared to those of the peptide TALRIRATYGEY (SEQ ID NO: 17)

### Example 11: Wound Closure Test of a Sheet of Freshly Isolated Primary Keratinocytes for the Peptides TALRIRATYGEY (SEQ ID NO: 17), LRIRATYG (SEQ ID NO: 13), LRIRFTYK (SEQ ID NO: 1), CSGLRIRFTYKSGC (SEQ ID NO: 3), and CLRIRFTYKC (SEQ ID NO: 2)

The wound closure test was carried out according to the protocol described above (Example 1) with freshly isolated primary keratinocytes. The peptide TALRIRATYGEY (SEQ ID NO: 17) was used as a control peptide, the recombinant human Epidermal Growth Factor (EGF) (Peprotech, France) as a positive control, and the culture medium alone as a negative control.

The peptides tested are:

(SEQ ID NO: 13)
LRIRATYG
(SEQ ID NO: 1)
LRIRFTYK
(SEQ ID NO: 2)
CLRIRFTYKC
(SEQ ID NO: 3)
CSGLRIRFTYKSGC

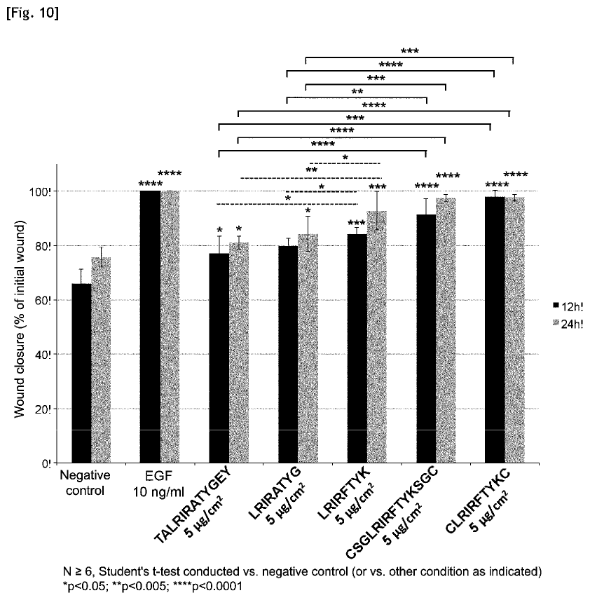

The results are presented in FIG. 10.

The results show significant greater activity to the peptides TALRIRATYGEY (SEQ ID NO: 17) and LRIRATYG (SEQ ID NO: 13) for all the peptides studied at 12 and 24 h. The peptides with the most significant activities are the peptides, in increasing order, LRIRFTYK (SEQ ID NO: 1), CSGLRIRFTYKSGC (SEQ ID NO: 3), and CLRIRFTYKC (SEQ ID NO: 2).

### Example 8: Examples of Compositions Comprising the Peptides According to the Invention

### Composition No. 1: W/O Emulsion (Water in Oil)

| Ingredients | % |
|---|---|
| AQUA (WATER) | QS 100 |
| HEXYL LAURATE | 37.000 |
| GLYCERIN | 5.000 |
| METHYL GLUCOSE ISOSTEARATE | 3.500 |
| DIMETHICONE | 3.000 |
| DISTEARDIMONIUM HECTORITE | 3.000 |
| EUPHORBIA CERIFERA (CANDELILLA) WAX | 1.500 |
| PEG-45/DODECYL GLYCOL COPOLYMER | 1.000 |
| SODIUM SALICYLATE | 0.580 |
| SODIUM BENZOATE | 0.480 |
| MAGNESIUM SULFATE | 0.100 |
| DISODIUM EDTA | 0.100 |
| PEPTIDE LRIRFTYK (SEQ ID NO: 1) | 0.0001 |

### Composition No. 2: O/W Emulsion (Oil in Water)

| Ingredients | % |
|---|---|
| AQUA (WATER) | QS 100 |
| CYCLOMETHICONE | 4.000 |
| GLYCERIN | 3.000 |
| HYDROGENATED POLYISOBUTENE | 3.000 |
| PPG-15 STEARYL ETHER | 2.000 |
| ETHYLHEXYL PALMITATE | 2.000 |
| STEARETH-2 | 2.000 |
| STEARETH-21 | 2.000 |
| STEARIC ACID | 1.750 |
| CETYL ALCOHOL | 1.500 |
| SODIUM SALICYLATE | 0.600 |
| POLYACRLAMIDE, C13-14 ISOPARAFFIN, | 0.400 |
| LAURETH-7 | |
| XANTHAN GUM | 0.300 |
| SODIUM BENZOATE | 0.200 |
| PEPTIDE CLRIRFTYKC (SEQ ID NO: 2) | 0.001 |

### Composition No. 3: Lotion

| Ingredients | % |
|---|---|
| AQUA (WATER) | QS 100 |
| GLYCERIN | 5.000 |
| SODIUM BENZOATE | 0.700 |
| SODIUM SALICYLATE | 0.550 |
| BIS-PEG-18 METHYL ETHER DIMETHYL | 0.500 |
| SILANE | |
| TETRASODIUM EDTA | 0.200 |
| PEPTIDE CSGLRIRFTYKSGC (SEQ ID NO: 3) | 0.003 |

### Composition No. 4: Gel

| Ingredients | % |
|---|---|
| AQUA (WATER) | QS 100 |
| CYCLOMETHICONE | 4.000 |
| GLYCERIN | 3.000 |
| SODIUM SALICYLATE | 0.600 |
| POLYACRLAMIDE, C13-14 ISOPARAFFIN, | 0.500 |
| LAURETH-7 | |
| CARBOMER | 0.500 |
| SODIUM BENZOATE | 0.200 |
| PEPTIDE LRIRFTYKSGLRIRFTYK (SEQ ID NO: 4) | 0.003 |

1. A peptide consisting of the sequence: LRIRX₁TYX₂, where:
X₁ is selected from amino acids A or F; and
X₂ is selected from amino acids G or K.

2. The peptide according to claim 1, characterized in that it has the following sequence: LRIRFTYK (SEQ ID NO: 1)

3. A peptide consisting of the sequence: CLRIRX₁TYX₂C, where:
X₁ is selected from amino acids A or F; and
X₂ is selected from amino acids G or K.

4. The peptide according to claim 2, characterized in that it has the following sequence: CLRIRFTYKC (SEQ ID NO: 2).

5. A peptide consisting of the sequence: CSGLRIRX₁TYX₂SGC, where:
X₁ is selected from amino acids A or F; and
X₂ is selected from amino acids G or K.

6. The peptide according to claim 3, characterized in that it has the following sequence: CSGLRIRFTYKSGC (SEQ ID NO: 3).

7. The peptide according to claim 1 for use as a medication.

8. The peptide according to claim 7, for use as a medication in:
the reinforcement of the dermal-epidermal junction, the cell-matrix adhesion of the skin; and/or
treating impairments of skin induced by dermatological pathologies, and/or
treating impairments of skin weakened by cosmetic or therapeutic treatment; and/or
the curative or preventive treatment of skin aging such as wrinkles, loosening, loss of elasticity, reduced healing, senile xerosis, modifications of the pigment system, depletion of the vascular network of the skin, irregularity of the skin grain, skin atrophy and alteration of the skin appendages.

9. A non-therapeutic cosmetic composition characterized in that it comprises at least one peptide according to claim 1.

10. The cosmetic composition according to claim 9, characterized in that it comprises from 0.00002% to 5%, preferably from 0.00005% to 0.1% and even more preferentially from 0.0001% to 0.001% by weight of the peptide and at least one cosmetically acceptable excipient.


SEQUENCE LISTING (23 sequences)

SEQ ID NO: 1 - protein, 8 aa, unidentified
  source 1..8: note = Other sequences
LRIRFTYK

SEQ ID NO: 2 - protein, 10 aa, unidentified
  source 1..10: note = Other sequences
CLRIRFTYKC

SEQ ID NO: 3 - protein, 14 aa, unidentified
  source 1..14: note = Other sequences
CSGLRIRFTYKSGC

SEQ ID NO: 4 - protein, 18 aa, unidentified
  source 1..18: note = Other sequences
LRIRFTYKSGLRIRFTYK

SEQ ID NO: 5 - protein, 8 aa, unidentified
  source 1..8: note = Other sequences
LRIRFTYG

SEQ ID NO: 6 - protein, 10 aa, unidentified
  source 1..10: note = Other sequences
CLRIRFTYGC

SEQ ID NO: 7 - protein, 14 aa, unidentified
  source 1..14: note = Other sequences
CSGLRIRFTYGSGC

SEQ ID NO: 8 - protein, 18 aa, unidentified
  source 1..18: note = Other sequences
LRIRFTYGSGLRIRFTYG

SEQ ID NO: 9 - protein, 8 aa, unidentified
  source 1..8: note = Other sequences
LRIRATYK

SEQ ID NO: 10 - protein, 10 aa, unidentified
  source 1..10: note = Other sequences
CLRIRATYKC

SEQ ID NO: 11 - protein, 14 aa, unidentified
  source 1..14: note = Other sequences
CSGLRIRATYKSGC

SEQ ID NO: 12 - protein, 18 aa, unidentified
  source 1..18: note = Other sequences
LRIRATYKSGLRIRATYK

SEQ ID NO: 13 - protein, 8 aa, unidentified
  source 1..8: note = Other sequences
LRIRATYG

SEQ ID NO: 14 - protein, 10 aa, unidentified
  source 1..10: note = Other sequences
CLRIRATYGC

SEQ ID NO: 15 - protein, 14 aa, unidentified
  source 1..14: note = Other sequences
CSGLRIRATYGSGC

SEQ ID NO: 16 - protein, 18 aa, unidentified
  source 1..18: note = Other sequences
LRIRATYGSGLRIRATYG

SEQ ID NO: 17 - protein, 12 aa, unidentified
  source 1..12: note = Other sequences
TALRIRATYGEY

SEQ ID NO: 18 - protein, 16 aa, unidentified
  source 1..16: note = Other sequences
LRIRATYGLRIRATYG

SEQ ID NO: 19 - protein, 8 aa, unidentified
  source 1..8: note = Other sequences
LRIRKTYG

SEQ ID NO: 20 - protein, 8 aa, unidentified
LRIRXTYX

SEQ ID NO: 21 - protein, 10 aa, unidentified
CLRIRXTYXC

SEQ ID NO: 22 - protein, 14 aa, unidentified
CSGLRIRXTYXSGC

SEQ ID NO: 23 - protein, 18 aa, unidentified
LRIRXTYXSGLRIRXTYX